(19) United States
(12) Patent Application Publication
(10) Pub. No.: US 2023/0190343 A1
(43) Pub. Date: Jun. 22, 2023

(54) OSTEOSYNTHESIS DEVICE COMPRISING AT LEAST ONE FIXATION PIN

(71) Applicant: NEWCLIP INTERNATIONAL, Luxembourg (LU)

(72) Inventors: Julien BALLERINI, Nantes (FR); Jean-Pierre PODGORSKI, Saint Crespin Sur Moine (FR); Grégoire LARCHE, Cholet (FR); Mark ROSS, Brisbane, Queensland (AU)

(21) Appl. No.: 18/018,128

(22) PCT Filed: Jul. 26, 2021
(86) PCT No.: PCT/EP2021/070833

§ 371 Date: Jan. 26, 2023

(30) Foreign Application Priority Data
Jul. 27, 2020 (FR) FR2007917

(51) Int. Cl. A61B 17/80; A61B 17/72

(52) U.S. Cl. CPC A61B 17/80; A61B 17/72; A61B 17/846

(57) ABSTRACT

Disclosed is an osteosynthesis device including at least one fixation pin suitable for being implanted in a bone material so as to ensure an at least partial reduction of a bone fracture, which osteosynthesis device includes a locking unit designed to lock in position the fixation pin implanted in the bone material. The locking unit can include: a support structure provided with a unit that enables its fixing on the bone material, and including a locking aperture suitable for being passed through by the fixation pin; and a clamping designed to be fitted in the locking aperture and to come into abutment against a part of the fixation pin in order to ensure its locking in position.

### BACKGROUND OF THE INVENTION

### Field of the Invention

The present invention relates to the general field of surgical equipment for osteosynthesis techniques.

It more particularly relates to the osteosynthesis devices comprising one or several fixation pins suitable for the reduction of a bone fracture.

### Description of the Related Art

The reduction and stabilisation of bone fractures are generally made by means of fixation screws and/or fixation pins implanted into the bone material, possibly in association with an osteosynthesis plate.

The fixation pins used are in the form of plain or threaded metal rods, at least one end of which is pointed or tapered to allow its driving into the bone material, generally by means of a surgical motor. They are generally used to stabilize the small-size bone fragments.

However, after having been driven, these pins may migrate within the receiving bone material, in either direction, depending on the stresses.

This possibility of movement may raise reduction stabilization problems and cause damages to the surrounding tissues.

To search to limit this possibility of displacement, after positioning of the pin, the surgeon may fold the protruding end thereof against the bone material or against the associated osteosynthesis plate.

However, such a folding or bending operation is not easy to carry out; moreover, it can prevent pin migration in only one direction.

### SUMMARY OF THE INVENTION

In order to remedy this drawback of the state of the art, the present invention proposes an osteosynthesis device comprising at least one fixation pin suitable for being implanted in a bone material so as to ensure an at least partial reduction of a bone fracture, said osteosynthesis device comprising locking means designed to lock in position said at least one fixation pin implanted into said bone material.

More particularly, the locking means of the osteosynthesis device according to the invention may comprise:
(a) a support structure provided with means for its fixation to the bone material, said support structure comprising a locking aperture through its thickness, said locking aperture being centred on an aperture axis and being delimited by an aperture contour, said locking aperture being adapted to be passed through by said at least one associated fixation pin, and
(b) a tightening means designed to be fitted into said locking aperture and to come into abutment against a part of said at least one fixation pin in order to ensure its locking in position.

According to a preferred embodiment, a part at least of said aperture contour of the locking aperture is provided with an aperture thread,
and said tightening means consists of a locking nut centred on a nut axis, said locking nut being delimited by — an upper nut part provided with a recess adapted for its driving into rotation by means of a suitable tool, — a lower nut part located at the opposite of said upper nut part, — an outer peripheral envelope comprising a nut thread adapted to cooperate with said aperture thread of said locking aperture of the support structure, and — an inner contour delimiting an axial hole adapted for the passage of said at least one fixation pin,
said locking nut still comprising a deformable tightening structure adapted to be deformed when said locking nut is screwed into said aperture thread and to come into abutment against a part of said at least one fixation pin housed within said axial hole.

Other non-limiting and advantageous features of the osteosynthesis device according to the invention, taken individually or according to all the technically possible combinations, are the following:
the tightening structure comprises at least one tightening tab arranged at the lower nut part;
said at least one tightening tab is deformable by bending about a bending axis perpendicular to said nut axis;
the tightening structure comprises a plurality of tightening tabs regularly distributed over the periphery of the lower nut part, each of said tightening tabs being deformable by bending about a bending axis perpendicular to said nut axis, the different bending axes of said tightening tabs being located in a same plane perpendicular to said nut axis;
the tightening structure comprises 2 to 4 tightening tabs, preferably 3;
the tightening structure comprises a plurality of tightening tabs separated from each other by slots extending in radial planes with respect to said nut axis;
the aperture contour of the locking aperture of the support structure has a generally frustoconical shape, the angle of said truncated cone with respect to the aperture axis of the locking aperture being between 9° and 11°;
the outer peripheral envelope of the locking nut forming the tightening means has a generally frustoconical shape, the angle of said truncated cone with respect to the nut axis of the locking nut being between 7° and 9°;
the means that allow the fixation of the support structure to the bone material consist of at least one fixation aperture arranged on said support structure, cooperating with a fixation screw;
the support structure is in the form of an osteosynthesis plate comprising (a) a plurality of fixation apertures, and (b) a plurality of locking apertures at least some of which are adapted to receive a fixation pin.

Preferably, the fixation apertures and the locking apertures of the osteosynthesis plate are identical so as to each receive either a fixation screw or a fixation pin.

Obviously, the different features, alternatives and embodiments of the invention can be associated with each other according to various combinations, insofar as they are not incompatible or exclusive with respect to each other.

### BRIEF DESCRIPTION OF THE DRAWINGS

Moreover, various other features of the invention emerge from the appended description made with reference to the drawings that illustrate non-limiting embodiments of the invention, and wherein:

FIG. 1 is a schematic cross-sectional view showing an osteosynthesis device according to the invention, before the screwing of the locking nut into its receiving aperture of the support structure to lock the fixation pin in position;

FIG. 2 is a schematic cross-sectional view, similar to FIG. 1, showing the osteosynthesis device according to the invention, in an activated position, after the screwing and tightening of the locking nut into its receiving aperture of the support structure to lock the fixation pin in position;

FIG. 3 is a perspective view of the locking nut of the osteosynthesis device according to the invention, in isolation, viewed from its upper nut part side;

FIG. 4 is a perspective view of the locking nut of FIG. 3, viewed from its lower nut part side;

FIG. 5 is a first side view of the locking nut illustrated in FIGS. 3 and 4;

FIG. 6 is a second side view of the locking nut illustrated in FIGS. 3 and 4;

FIG. 7 is a top view of the locking screw illustrated in FIGS. 3 to 6;

FIG. 8 shows the locking nut of the osteosynthesis device according to the invention, through the central hole of which extends a fixation pin, in a perspective view from the lower nut part side;

FIG. 9 is a view similar to FIG. 8 illustrating the locking nut and the fixation pin associated in a perspective view from the upper nut part side;

FIG. 10 is a perspective view that illustrates an osteosynthesis device according to the invention, whose support structure is in the form of an osteosynthesis plate, positioned on a receiving bone material;

FIG. 11 is an enlarged view of a part of FIG. 10.

### DESCRIPTION OF THE PREFERRED EMBODIMENTS

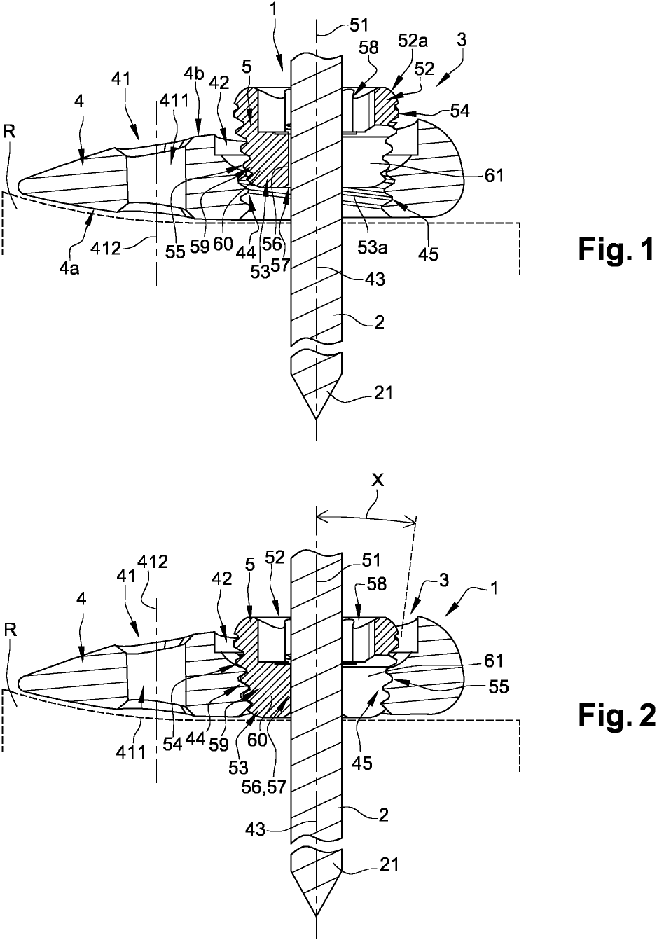

The osteosynthesis device 1 illustrated in FIGS. 1 and 2 comprises a fixation pin 2 adapted to be implanted in a bone material R so as to ensure an at least partial reduction of a bone fracture, and it also comprises locking means 3 arranged so as to lock this fixation pin 2 in position.

The fixation pin 2 is made of metal, for example stainless steel, titanium or cobalt-chromium. It is here in the form of a plain rod, having at least one point-shaped end 21; its length may be between 20 and 200 mm and its diameter, which is constant, may be between 0.8 and 4 mm.

The locking means 3 comprise:
(a) a support structure 4 provided with means 41 for its fixation to the bone material R, and comprising a locking aperture 42 through its thickness, said locking aperture 42 being centred on an aperture axis 43 and being delimited by an aperture contour 44, said locking aperture 42 being adapted to be passed through by the fixation pin 2, and
(b) a tightening means 5 designed to be fitted into said locking aperture 42 and to come into abutment against a part of said fixation pin 2 in order to ensure its locking in position.

The aperture contour 44 comprises an aperture thread 45, and the tightening means 5 consists of a locking nut 5 centred on a nut axis 51.

The support structure 4 is here in the form of an osteosynthesis support plate of a few millimetres thick and that is delimited by a lower face 4a intended to come into contact with the receiving bone material R and by an opposite upper face 4b.

In addition to the above-mentioned locking aperture 42 of the locking means 3, adapted to receive the fixation pin 2 and the tightening means 5, the support structure 4 is provided with at least one aperture 411 through its thickness, between its lower face 4a and its upper face 4b; this aperture 411 is intended for the passage of a fixation member 412 (in particular in the form of a fixation screw schematically shown in FIGS. 1 and 2) for its fixation to the receiving bone material R.

The aperture 411 and the associated fixation member 412 form the means 41 for the fixation of the osteosynthesis plate 4 on the bone material R.

The aperture contour 44 of the locking aperture 42 of the support structure 4 has a generally frustoconical shape, the apex of which is directed towards the lower face 4a of the support structure 4. The angle X of this truncated cone with respect to the aperture axis 43 is between 5° and 15° (preferably, this angle X is between 8° and 12°, and still preferably between 9° and 11°).

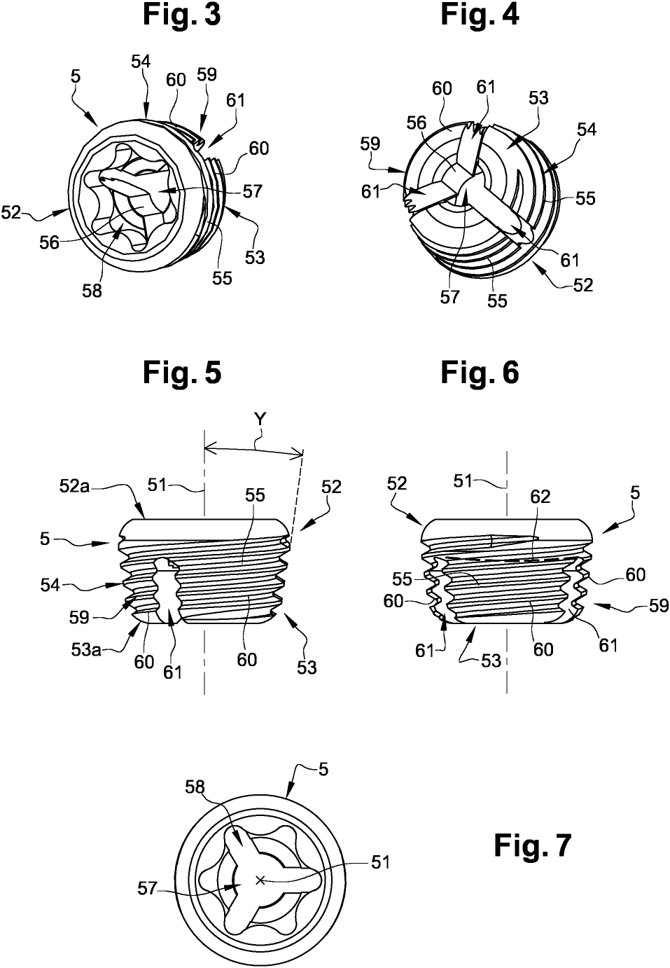

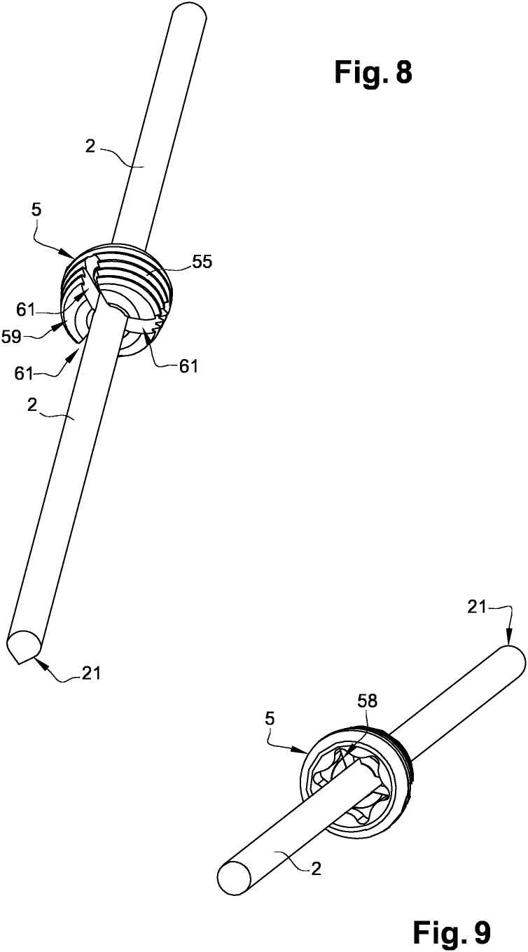

The locking nut 5 is illustrated in isolation in FIGS. 3 to 7, and in association with a fixation pin 2 in FIGS. 8 and 9.

This locking nut 5 is delimited by — an upper nut part 52, comprising an upper nut end 52a, — a lower nut part 53, comprising a lower nut end 53a, located at the opposite of said upper nut part 52, — an outer peripheral envelope 54 comprising a nut thread 55 adapted to cooperate with the aperture thread 45 of the locking aperture 42, and — an inner contour 56 delimiting an axial hole 57 adapted for the passage of the fixation pin 2.

The upper nut part 52 is provided with a recess 58 adapted to be driven into rotation by means of an appropriate tool, for the screwing or unscrewing of the locking nut 5.

The outer peripheral envelope 54 of the locking nut 5 has a generally frustoconical shape, the apex of which is directed towards the lower nut part 53. The angle Y of this outer peripheral envelope 54 of frustoconical shape is between 4°and 12° (preferably, this angle Y is between 6° and 10°, and still preferably between 7° and 9°).

The locking nut 5 still comprises a deformable tightening structure 59, adapted to be deformed when said locking nut 5 is screwed into said aperture thread 45 and to come into abutment against a part of the fixation pin 2 housed within the axial hole 57.

This tightening structure 59 comprises at least one tightening tab 60, and preferably several tightening tabs 60, arranged at the lower nut part 53.

Herein, the tightening structure 59 comprises a plurality of tightening tabs 60 regularly distributed over the periphery of the lower nut part 53. These tightening tabs 60 are separated from each other by slots 61 that extend in radial planes with respect to the nut axis 51; and each of these tightening tabs 60 are deformable by bending about a bending axis perpendicular to said nut axis 51.

The slots 61 extend from the lower nut end 53a, over part of the height of the locking nut 5, herein, just over half the height of the locking nut 5.

The corresponding bending axis is illustrated in FIG. 6 by the reference 62.

The different bending axes 62 of the tightening tabs 60 are located in a same plane perpendicular to said nut axis 51.

Preferably, the tightening tabs 60 are 2, 3 or 4 in number. In the embodiment illustrated, the tightening structure 59 comprises 3 tightening tabs 60.

The radial slots 61 made in the lower nut part 53 define the tightening tabs 60, each deformable about a bending axis 62 perpendicular to the nut axis 51. For each tightening tab 60, the bending axis 62 corresponds to the line that connects the bottom to the two radial slots 61 arranged on either side of it.

The nut thread 55 extends over the whole or almost the whole height of the outer peripheral envelope 54, between the upper nut part 52 and the lower nut part 53.

The tightening tabs 60 are integral with the locking nut 5. Their outer contour extends over the outer peripheral envelope 54 of frustoconical shape, and this outer contour of the tightening tabs 60 comprises a part of the nut thread 55.

The shape of the locking aperture 42 is adapted to apply a radial pressure force to the contour of the lower part 53 of the locking nut 5 as the screwing of the latter on the aperture thread 45 goes along, in such a way as to obtain the desired tightening function.

For that purpose, before the screwing, the diameter of the lower part of the locking aperture 42 is lower than the diameter of the lower end 53a of the locking nut 5.

Before activation of the tightening structure 59, the axial hole 57 of the locking nut 5 allows the passage through it of a fixation pin 2.

The axial hole 57 may then be in the form of a channel or a cylindrical aperture whose diameter corresponds, with a clearance, to the diameter of the fixation pin 2.

This passage or sliding possibility is illustrated in FIG. 1, which shows the locking nut 5 before its tightening into the locking aperture 42, by the small space present between the inner contour 56 of the locking nut 5 (that delimits the axial hole 57) and the cylindrical wall opposite the fixation pin 2.

In FIGS. 8 and 9, the locking nut 5 is not introduced into a locking aperture 42 of a support structure 4, and the fixation pin 2 may also slide freely in the axial hole 57 of said locking nut 5.

Once the support structure 4 fixed to the receiving bone material R, with the locking nut 5 not screwed or not fully screwed into the locking aperture 42 (as illustrated in FIG. 1, for example), the fixation pin 2 may be positioned by the surgeon, passing through the axial hole 57 of the locking nut 5, in such a way as to perform the desired reduction of the bone fracture.

When the fixation pin 2 is correctly positioned within the bone material R, its positioning may be locked by screwing the locking nut 5 on the thread of the locking aperture 42 of the support structure 4.

During this screwing, the suitable shape of the locking hole 42 forces the tightening tabs 60 of the locking nut 5 to be radially deformed towards the nut axis 51, and hence towards the fixation pin 2.

This deformation is made by bending of the tightening tabs 60 about their respective bending axis 62, until these tightening tabs 60 come in abutment against the fixation pin 2.

It is then understood that a forced screwing of the locking nut 5 allows the tightening tabs 60 to apply a high pressure against the fixation pin 2, ensuring the locking in position of this fixation pin 2 that can no longer slide, or migrate, in either direction, as illustrated in FIG. 2.

The plurality of tightening tabs 60 regularly distributed over the periphery of the lower nut part 53 makes is possible to obtain a homogeneous tightening of the fixation pin 2.

The locking nut 5 acts as a kind of locking wedge between the aperture contour 44 and the fixation pin 2.

The shape of the aperture contour 44 of the locking aperture 42 (angle X) and the shape of the outer peripheral envelope 54 of the locking nut 5 (angle Y), as well as their size, are adapted to obtain the deformation of the tightening tabs 60 and the desired locking, when the locking nut 5 is screwed into the locking aperture 42.

In the embodiment illustrated, the aperture contour 44 of the locking aperture 42, and the outer peripheral envelope 54 of the locking nut 5 both have a generally frustoconical shape, but with a different apex angle; in an alternative embodiment, the outer peripheral envelope of the locking nut may be of generally cylindrical shape, the aperture contour of the locking aperture of the support structure having a generally frustoconical shape.

As another alternative, other means than a locking screw may be used to block the fixation pin with respect to the support structure 4. For example, a conical crown may be provided, comprising at least a flexible portion, capable of being impacted between the aperture contour 44 and the fixation pin 2, thus forming a kind of locking wedge.

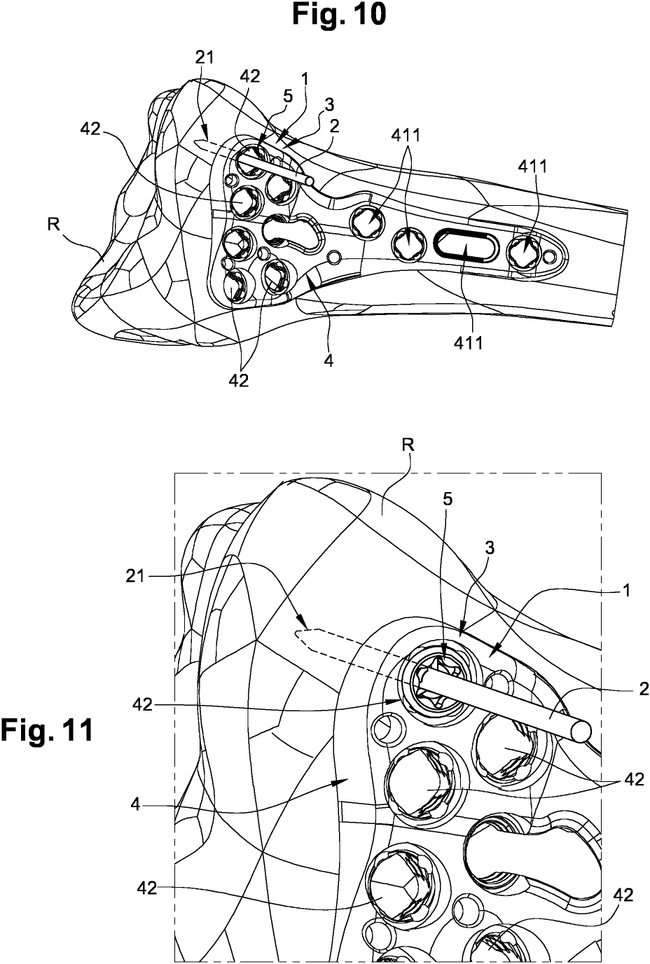

As illustrated in FIGS. 10 and 11, the support structure 4 can be in the form of an osteosynthesis plate comprising a plurality of fixation apertures 411, for its fixation on the bone material R, and a plurality of locking apertures 42 each adapted to receive a fixation pin 2.

In a particular embodiment, the fixation apertures for the fixation on the bone material R, and the locking apertures for receiving the fixation pins 2, of the osteosynthesis plate 4, are planned identical so as to each accommodate either a fixation screw 412 or a fixation pin 2.

It is understood that the surgeon can reduce a bone fracture by means of one or several fixation pins 2, in a conventional manner, and lock each of these fixation pins in position when he estimates that their position is correct.

The fixation pins 2 may be fixed temporarily or permanently into the receiving bone material.

After a temporary fixation, the surgeon unscrews the locking nut 5; and the fixation pin 2 then recovers its ability to slide in the axial hole 57 of the locking nut 5, for its removal.

For a durable fixation, the surgeon cuts the fixation pin 2 flush with the support plate to avoid the presence of a protruding part.

1. An osteosynthesis device comprising at least one fixation pin suitable for being implanted in a bone material so as to ensure an at least partial reduction of a bone fracture,
wherein the osteosynthesis device comprises locking means designed to lock in position said at least one fixation pin implanted into said bone material.

2. The osteosynthesis device according to claim 1, wherein said locking means comprise:
a support structure provided with means for the support structure’s fixation to the bone material, said support structure comprising a locking aperture through the support structure’s thickness, said locking aperture being centred on an aperture axis and being delimited by an aperture contour, said locking aperture being adapted to be passed through by said at least one associated fixation pin, and
a tightening means designed to be fitted into said locking aperture and to come into abutment against a part of said at least one fixation pin in order to ensure its locking in position.

3. The osteosynthesis device according to claim 2, wherein a part at least of said aperture contour is provided with an aperture thread,
and wherein said tightening means consists of a locking nut centred on a nut axis, said locking nut being delimited by
an upper nut part provided with a recess adapted for the upper nut part’s driving into rotation by means of a suitable tool,
a lower nut part located at the opposite of said upper nut part,
an outer peripheral envelope (54) comprising a nut thread adapted to cooperate with said aperture thread of said locking aperture, and
an inner contour delimiting an axial hole adapted for the passage of said at least one fixation pin,
said locking nut still comprising a deformable tightening structure adapted to be deformed when said locking nut is screwed into said aperture thread and to come into abutment against a part of said at least one fixation pin housed within said axial hole.

4. The osteosynthesis device according to claim 3, wherein said tightening structure comprises at least one tightening tab arranged at said lower nut part.

5. The osteosynthesis device according to claim 4, wherein said at least one tightening tab is deformable by bending about a bending axis perpendicular to said nut axis.

6. The osteosynthesis device according to claim 5, wherein said tightening structure comprises a plurality of tightening tabs regularly spaced apart over the periphery of said lower nut part, each of said tightening tabs being deformable by bending about a bending axis perpendicular to said nut axis, the different bending axes of said tightening tabs being located in a same plane perpendicular to said nut axis.

7. The osteosynthesis device according to claim 6, wherein said tightening structure comprises 2 to 4 tightening tabs).

8. The osteosynthesis device according to claim 4, wherein said tightening structure comprises a plurality of tightening tabs separated from each other by slots extending in radial planes with respect to said nut axis.

9. The osteosynthesis device according to claim 2, wherein the aperture contour of the locking aperture of the support structure has a generally frustoconical shape, the angle of said truncated cone with respect to the aperture axis of said locking aperture being between 9° and 11°.

10. The osteosynthesis device according to claim 3, wherein the outer peripheral envelope of the locking nut forming said tightening means has a generally frustoconical shape, the angle of said truncated cone with respect to the nut axis of said locking nut being between 7° and 9°.

11. The osteosynthesis device according to claim 2, wherein said means (41) that allow the fixation of the support structure to the bone material consist of at least one fixation aperture arranged on said support structure, cooperating with a fixation screw.

12. The osteosynthesis device according to claim 11, wherein said support structure is in the form of an osteosynthesis platecomprising a plurality of fixation apertures and a plurality of locking apertures at least some of which are adapted to receive a fixation pin.

13. The osteosynthesis device according to claim 12, wherein said fixation apertures and said locking apertures of the osteosynthesis plate are identical so as to each accommodate either a fixation screw or a fixation pin.

14. The osteosynthesis device according to claim 6, wherein said tightening structure comprises 3 tightening tabs.

15. The osteosynthesis device according to claim 9, wherein the outer peripheral envelope of the locking nut forming said tightening means has a generally frustoconical shape, the angle of said truncated cone with respect to the nut axis of said locking nut being between 7° and 9°.

16. The osteosynthesis device according to claim 3, wherein the aperture contour of the locking aperture of the support structure has a generally frustoconical shape, the angle of said truncated cone with respect to the aperture axis of said locking aperture being between 9° and 11°.

17. The osteosynthesis device according to claim 4, wherein the aperture contour of the locking aperture of the support structure has a generally frustoconical shape, the angle of said truncated cone with respect to the aperture axis of said locking aperture being between 9° and 11°.

18. The osteosynthesis device according to claim 5, wherein the aperture contour of the locking aperture of the support structure has a generally frustoconical shape, the angle of said truncated cone with respect to the aperture axis of said locking aperture being between 9° and 11°.

19. The osteosynthesis device according to claim 6, wherein the aperture contour of the locking aperture of the support structure has a generally frustoconical shape, the angle of said truncated cone with respect to the aperture axis of said locking aperture being between 9° and 11°.

20. The osteosynthesis device according to claim 7, wherein the aperture contour of the locking aperture of the support structure has a generally frustoconical shape, the angle of said truncated cone with respect to the aperture axis of said locking aperture being between 9° and 11°.
